(19) United States
(12) Patent Application Publication
(10) Pub. No.: US 2023/0117122 A1
(43) Pub. Date: Apr. 20, 2023

(54) BIOFILM INHIBITION SYSTEM BASED ON UV-LEDS

(71) Applicants: Milan Minsky, Newton, MA (US); Julie Fouquet, Portola Valley, CA (US); Iosif Izrailit, Newton, MA (US); Rei Landsberger, Santa Barbara, CA (US)

(72) Inventors: Milan Minsky, Newton, MA (US); Julie Fouquet, Portola Valley, CA (US); Iosif Izrailit, Newton, MA (US); Rei Landsberger, Santa Barbara, CA (US)

(21) Appl. No.: 17/965,126

(22) Filed: Oct. 13, 2022

Related U.S. Application Data
(60) Provisional application No. 63/256,692, filed on Oct. 18, 2021.

(51) Int. Cl. A61L 2/10; A61L 2/28; C02F 1/32

(52) U.S. Cl. CPC A61L 2/10; A61L 2/28; C02F 1/325; C02F 2103/08

(57) ABSTRACT

A biofilm inhibition system, for a sensor which includes a chamber with an interior volume and an interior surface exposed to direct contact with a fluid during sensor operation, includes an ultraviolet light emitting diode (UV-LED) and an optical subsystem. The optical subsystem is coupled to the UV-LED to deliver a portion of the emitted UV light to illuminate a substantial fraction of the interior volume and the interior surface. A watertight housing encloses the UV-LED and at least a portion of the optical subsystem.

### CROSS REFERENCE TO RELATED APPLICATION

This application claims the benefit of U.S. Provisional Patent Application Ser. No. 63/256,692, filed on Oct. 18, 2021, which is hereby incorporated by reference as if set forth in full in this application for all purposes.

This invention was made with US Government support under Federal Award DE-SC0020921 awarded by the Department of Energy. The Government has certain rights in this invention.

### BACKGROUND

Marine sensors are subject to biofouling, a term indicative of negative impacts on the operation of those sensors due to the gradual accumulation of living micro- and macro-organisms on exposed surfaces, including those of electrodes essential to carry out the sensing. A variety of organisms, typically including bacteria, fungi, protozoa, algae and invertebrates, are generally present in aqueous fluids including the sea water or freshwater that these sensors are intended to operate in. The introduction of any object with an accessible surface into a body of water containing such organisms almost invariably initiates the formation of a biofilm composed of bacteria embedded in a slimy matrix of extracellular secretions. The biofilm typically spreads and thickens over time, encouraging larger micro-organisms and ultimately macro-organisms such as marine worms, for example, to adhere in turn, forming a multi-species community.

This sequence of events can be particularly problematic for sensors such as conductivity-temperature (CT) sensors, conductivity-temperature-depth (CTD) sensors, dissolved oxygen sensors, and others that require entry of a fluid that must come into contact with internal surfaces, sometimes for prolonged periods. Electrical parameter measurements made by these sensors could be subject to significant errors when biofilms form on those surfaces, even if matters don't progress to the extreme case where fluid flow through the sensor chamber is blocked by a “plug” of bio-matter. In current practice, measures are generally taken to minimize biofouling, either by chemically inhibiting the initial formation and subsequent growth of biofilms, or by frequent inspection and cleaning to remove the films at an early stage, before they cause too much trouble.

Tributyltin oxide, a chemical once widely used in the former approach, usually in the form of a slowly dissolving block surrounding the sensor housing, works essentially by poisoning the water in the vicinity of the sensors. This is clearly an undesirable approach, even though it is still technically permitted for oceanographic research. The block gradually dissolves and dissipates in the fluid and requires replenishing. This approach, therefore, also requires periodic visits to the sensor deployment sites. The inconvenience and expense incurred in having to inspect and clean or replace sensors at frequent intervals is a burden. Biofilms can gain a substantial hold in as short a time as a few weeks, and although a timeline of months is more typical, the need to visit monitoring sites even twice a year in remote locations, and/or at significant depths below the water surface is a problem.

There is therefore a need for systems that could significantly inhibit formation and growth of biofilms on sensitive surfaces within sensors with minimal harm to the local environment. Ideally, the systems would be compact, inexpensive, have low power consumption, and could either be retrofitted to conventional sensors or integrated into them during fabrication.

### SUMMARY

The present invention includes systems and methods for inhibiting the formation and growth of biofilms within sensors exposed to fluids that contain microorganisms. In one embodiment, a biofilm inhibition system (BIS), for a sensor comprising a sensor chamber with an interior volume and an interior surface exposed to direct contact with a fluid during sensor operation, comprises: an ultraviolet light emitting diode (UV-LED); and an optical subsystem coupled to the UV-LED to deliver a portion of the emitted UV light to illuminate a substantial fraction of the interior volume and the interior surface of the sensor chamber; wherein a watertight housing encloses the UV-LED and at least a portion of the optical subsystem.

In another embodiment, a biofilm inhibition system (BIS), for a sensor comprising a chamber with an interior volume and an interior surface exposed to direct contact with a fluid during sensor operation, comprises: a fluid treatment chamber; and an ultraviolet light emitting diode (UV-LED). The fluid treatment chamber has an input port for the entry of untreated fluid and an output port from which treated fluid may be pumped from the treatment chamber to enter the sensor chamber. The UV-LED is configured to illuminate the fluid within the fluid treatment chamber. A watertight housing encloses the UV-LED.

In yet another embodiment, a method of inhibiting development of a biofilm in a sensor that comprises a chamber with an interior volume and an interior surface exposed to direct contact with a fluid during sensor operation comprises: capturing emission from an ultraviolet light emitting diode (UV-LED); and using an optical subsystem coupled to the UV-LED to deliver a portion of the emitted UV light to illuminate a substantial fraction of the interior volume and the interior surface of the sensor chamber. A watertight housing encloses the UV-LED and at least a portion of the optical subsystem.

### BRIEF DESCRIPTION OF THE DRAWINGS

FIG. 1 illustrates a biofilm inhibition system (BIS) according to some embodiments of the present invention.

FIG. 2 illustrates how the UV output of a BIS according to embodiments shown in FIG. 1 may interact with fluid in a typical marine sensor.

FIG. 3 illustrates an arrangement of optical components within a BIS according to some embodiments shown in FIG. 1.

FIG. 4 illustrates a biofilm inhibition system (BIS) according to some other embodiments of the present invention.

FIG. 5 illustrates how the UV output of a BIS according to embodiments shown in FIG. 4 may interact with fluid in a typical marine sensor.

FIG. 6 illustrates an arrangement of optical components within a BIS according to some embodiments shown in FIG. 4.

### DETAILED DESCRIPTION OF EMBODIMENTS

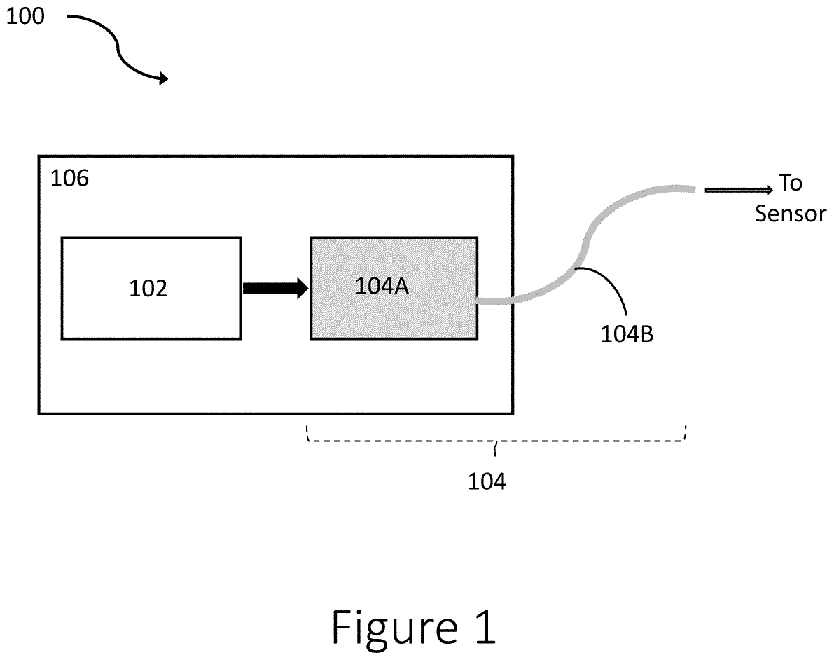

The manner in which the present invention provides its advantages can be more easily understood with reference to FIGS. 1 through 6.

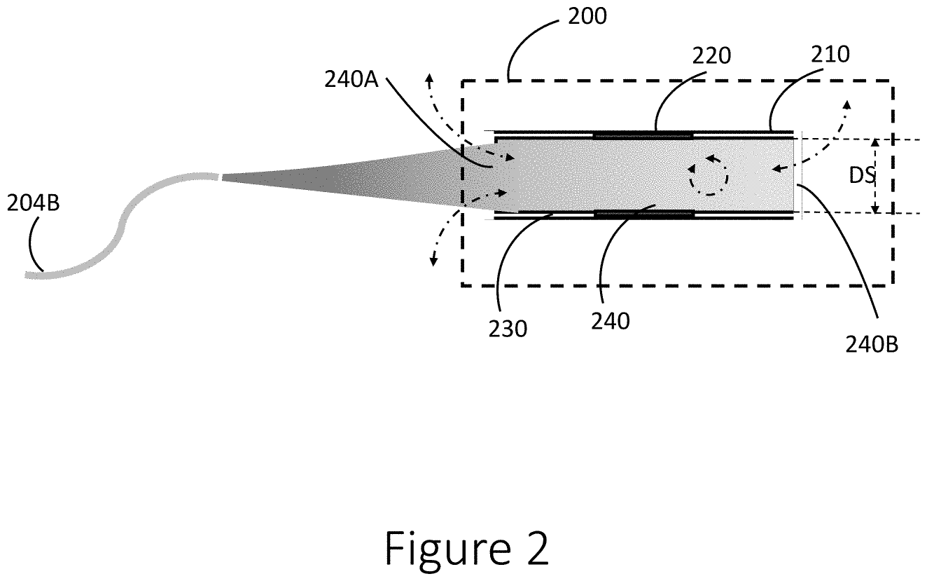

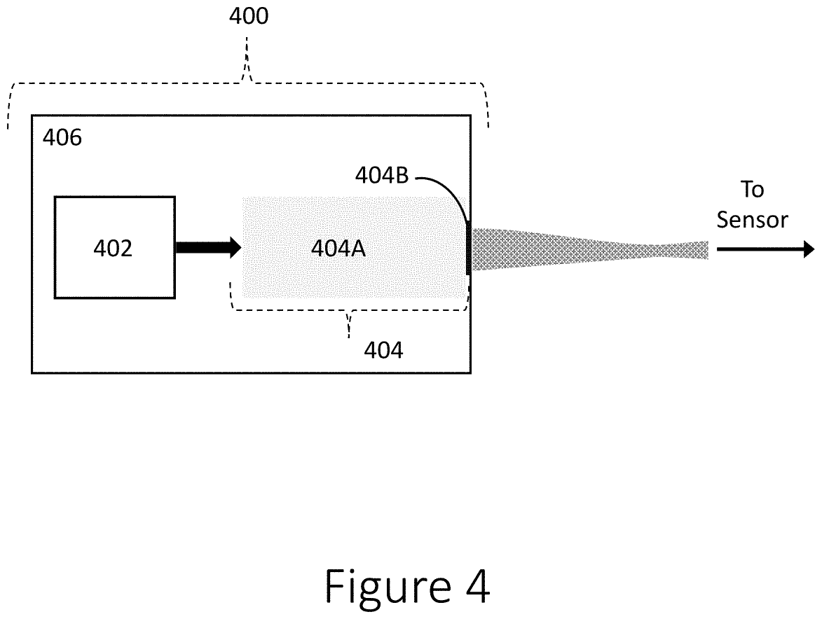

FIG. 1 illustrates a biofilm inhibition system (BIS) according to some embodiments of the present invention. The sensor type envisaged as the target for this system (and for the system shown in FIG. 4, for another set of embodiments) is a fluid sensor commonly termed a marine sensor. This typically includes a cylindrical sensing chamber, the internal volume of which is filled with the fluid of interest during sensor operation. Further details of such sensors will be described below in discussing FIGS. 2 and 4.

BIS 100 includes an ultraviolet light emitting diode (UV-LED) 102, an optical subsystem 104, and a housing 106. The optical subsystem includes one or more bulk optical components 104A and an optical fiber 104B. The combination captures a portion (typically on the order of 1% for currently available surface-emitting LEDs, potentially significantly higher for edge-emitting LEDs) of the UV emitted by the UV-LED 102 and transmits it through a currently available 1 mm fiber out of the BIS housing to ultimately reach a sensor (not shown in this figure). As will be explained in more detail below, the captured UV exits the fiber to enter the sensor chamber and there illuminates a substantial fraction of the interior volume of the chamber and the area of the interior surface of interest. The precise meaning of “substantial” in this context may vary according to the specific application, but in many cases can be taken to mean over 50% of the interior volume and over 50% of the area of the interior surface. Values of 100% may well be achievable.

The optical subsystem 104 may include some active components for optical alignment adjustments in some embodiments, but in a majority of cases will be entirely passive, with any necessary adjustments being performed manually, offline. In all the embodiments of FIG. 1, lens or lenses 104A and fiber 104B are selected in part on suitability for ultraviolet radiation transmission.

The micro-organisms present in biofilms are generally more easily killed by radiation in the UVC band, which is defined for the purposes of this application as the wavelength band between 200 nm and 300 nm, than in the longer wavelength (UVA and UVB) ultraviolet radiation bands. Accordingly, in some embodiments, UV-LED 102 in BIS 100 is a UVC-LED, emitting significant amounts of radiation in the UVC band. One type of UVC-LED commercially available has a peak emission wavelength of 285 nm, which is a particularly effective choice.

Housing 106, which encloses UV-LED 102, lens (or lenses) 104A, and an input face of fiber 104B, has to be watertight to protect the contents of the BIS, in particular to protect the UV-LED and associated electronic circuitry (not shown). It also has to be capable of withstanding external pressures likely to be encountered during operation. In many applications, the sensors are deployed well below the surface of the body of water. A typical maximum depth requirement is 200 m, as low light penetration at greater depths prevents biofouling being a problem.

In the case shown in FIG. 1, a short length of the fiber 104B is present within the housing, but in some other cases, the input end of the fiber may be flush with the housing

FIG. 2 is a cross-section side view illustrating how the UV output from a BIS such as BIS 100 may be directed to interact with fluid in the sensing chamber of a typical marine sensor in a way that inhibits biofilm formation on the interior surfaces of that chamber. The marine sensor is represented as element 200, including a cylindrical sensing chamber 210, which in practice may be much longer relative to its diameter than shown here for convenience. Other components present in sensor 200, not shown, include electronic circuitry for operating the sensing element or elements within the chamber, and a communication module for transmitting data collected or derived by those elements to a remote control and analysis node.

The sensing elements are shown here as two electrodes 220, set flush with the inner cylindrical surface 230 of the chamber. The surface 230, including the top surfaces of the electrodes, is therefore the target surface requiring protection from biofilm formation and growth. In some other embodiments, the electrodes may project beyond the cylindrical surface 230, exposing side edges to the fluid 240 within the chamber, in which case the total area of the electrode top surfaces and side surfaces require protection as well as the remaining inner cylindrical chamber wall. The same considerations apply to any other components projecting into the chamber, but in general, the inner wall is typically unbroken, allowing fluid to pass through with minimal obstruction.

The sensor and chamber have fluid entry ports 240A and 240B, though the direction of fluid flow shown from left to right is an arbitrary choice. In some marine sensors, the fluid is pumped through the chamber; in others it may simply flow in and out without external influence, beyond the disturbance of the sensor itself being present. Possible fluid flow paths are indicated by interrupted dashed lines. Some inner circulation loops may occur as shown, with corresponding effects on the time of fluid contact with surface 230.

Fiber 204B is shown with its output end face positioned slightly to the left of inlet port 240A such that the UV light exiting it emerges as a divergent beam, spreading out within the sensor chamber. A useful rule of thumb for a standard fiber with a numerical aperture of 0.22 is to position the fiber output end at a separation from the sensor of about twice the sensor chamber diameter, allowing the entire entrance to be filled by the beam emerging from the fiber end face, The shaded beam profile is a simplified representation, indicating a first order reduction in intensity with axial distance along the chamber. Scattering in the fluid and at surface 230 then acts to even out the distribution to some degree. The objective is to provide UV intensities close to the chamber's interior wall that can kill enough of the microorganisms that happen to be in the vicinity to either prevent biofilm formation altogether, or, more practically realizable, to keep their rate of growth low enough to significantly reduce the frequency at which sensor maintenance is required. The intensity required depends on the concentration and type of organisms anticipated at the sensor location, and these factors may vary seasonally, and during events like sudden algae blooms. Values of the order of 70 μW/cm² of UV at a peak wavelength of 285 nm have been found to be satisfactory in experimental conditions in the San Francisco Bay, Calif. and near Sequim Bay, Wash.

While the fiber is fixed in position slightly outside the sensor chamber in many embodiments, as shown in FIG. 2, alternative embodiments would be feasible if and when side-emitting fibers become readily available. A fiber of this type could be inserted deeper into the sensor chamber, delivering the UV in a more distributed fashion along the length of the chamber. In some embodiments, not shown, two fibers may be used, delivering UV to each end of the sensor chamber, producing a more even intensity distribution,

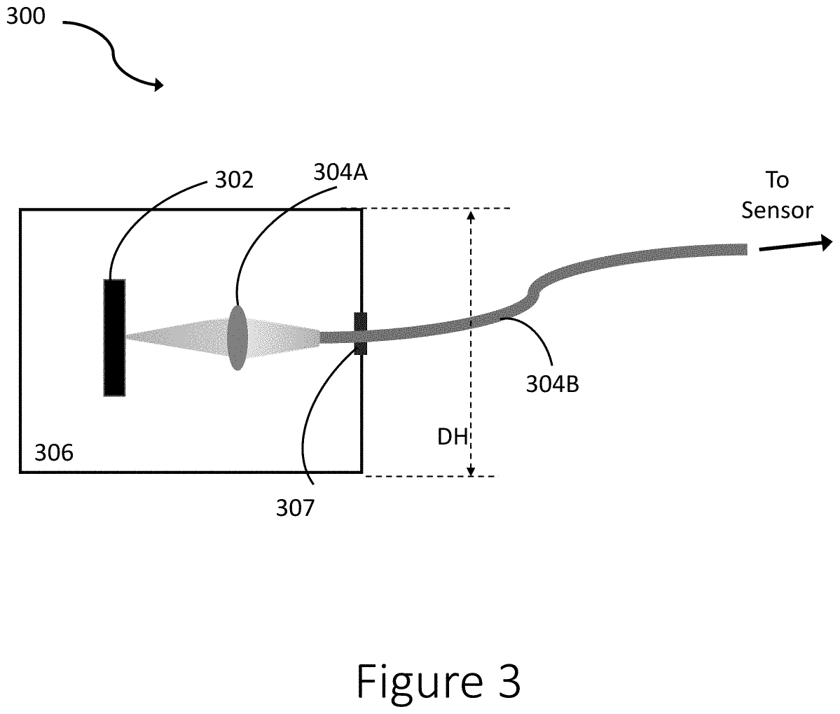

For applications where the sensor system doesn't use a pump to force water flow through the sensor chamber, it may be particularly desirable to minimize any fluid flow disturbance in the aqueous environment close to and within the sensor caused by the BIS. This allows measurement data gathered from the sensor to be taken as primarily reflective of intrinsic fluid characteristics (such as conductivity) untainted by the presence of any components of the BIS. Rough rules of thumb followed in such applications may put minimum thresholds on the optical distance between the sensor and any nearby object of significant size (such as a BIS housing) and on the fraction of cross-sectional area of the sensor chamber blocked for fluid flowing into the sensor chamber. In the embodiment shown in FIGS. 1-3, this may be achieved by keeping the BIS housing at a distance from the sensor at least equal to the diameter of the housing (indicated in FIG. 3 as DH) and keeping the diameter of fiber 204B small enough that the area blocked is no greater than 5% of the cross-sectional area of the chamber cavity. It is quite easy to satisfy the requirements for a typical marine sensor having a chamber with a length L of about 15 cm and an inner diameter DS of 5 mm, with a commercially available UV-transmitting optical fiber of diameter 1 mm and length 15 cm to 25 cm.

It may be helpful to provide some mechanical support for the fiber across the gap between BIS and sensor, one option being a cylindrical mesh with internal spokes holding the fiber axially while presenting minimal resistance to fluid flow. The choice of material for the mesh is important to prevent biofilm formation and growth on the mesh itself which could in turn disturb water flow in the vicinity of the sensor, or even block water from entering the sensor chamber. Copper is one reasonable choice; though it works through toxicity, it is not nearly as toxic as tributylin. Another good choice is Teflon, whose slippery surface discourages biofilm adhesion.

FIG. 3 illustrates an arrangement of the optical components of a BIS 300 that can provide fiber-guided UV radiation for a sensor as shown in FIG. 2. Element 302 is a UV-LED, typically mounted on a heat sink (not separately shown). The optical subsystem 304A capturing UV emitted by source 302 and coupling it into fiber 304B comprises one or more lenses, shown here as a single lens for simplicity. For a surface-emitting UV LED, efficiency of capture of the UV emission and delivery to the end of the fiber may be on the order of 1%. Fiber 304B passes through housing 306 at sealable exit port 307.

FIG. 4 illustrates a biofilm inhibition system (BIS) according to some other embodiments of the present invention that provide an unguided UV beam to the target sensor. BIS 400 is similar to BIS 100 of FIG. 1 in including an ultraviolet light emitting diode (UV-LED), an optical subsystem, and a housing, labeled 402, 404, and 406 respectively. A key difference is that in BIS 400 the housing encloses not only UV-LED 402, but the entirety of the optical subsystem 404, including bulk optical focusing elements 404A and a UV-transparent window 404B set into the housing wall. 404A is designed and positioned to capture a significant portion of the UV emitted by the UV-LED 402 and then shape the captured UV into a free space beam that exits the housing through window 404B to ultimately reach a sensor (not shown in this figure).

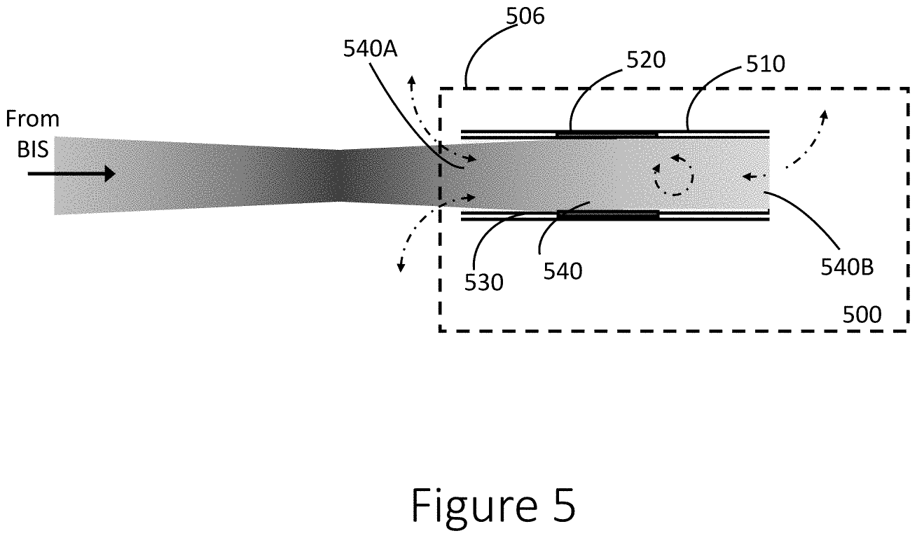

As will be described in more detail below with regard to FIG. 5, after entering the sensor and its sensing chamber, the UV provided by BIS 400 is able to illuminate a substantial fraction of the interior volume of the chamber and the interior surface of interest.

Other considerations discussed above with respect to the embodiments of FIG. 1 regarding using a UVC-LED, selecting optical components with high UV transmission, and the need for a watertight and pressure withstanding housing apply to the BIS embodiments of FIG. 4.

FIG. 5 is a cross-section side view illustrating how the UV output from a BIS such as BIS 400 may be directed to interact with fluid in the sensing chamber of a typical marine sensor in a way that inhibits biofilm formation on interior surfaces of that chamber. The marine sensor is represented as element 500, including a cylindrical sensing chamber 510. Other components present in sensor 500, not shown, include electronic circuitry for operating the sensing element or elements within the chamber, and a communication module for transmitting data collected or derived by those elements to a remote control and analysis node.

The sensing elements are shown here as two electrodes 520, set flush with the inner cylindrical surface 530 of the chamber. This surface 530, including the top surfaces of the electrodes, is therefore the “target” surface requiring protection from biofilm formation and growth. In some other embodiments, the electrodes may project beyond the cylindrical surface 530, exposing side edges to the fluid 540 within the chamber, in which case the electrode top surfaces and side surfaces require protection as well as the remaining cylindrical inner wall of the chamber. The exposed surfaces of any other components present in the chamber would also be protected, but in general, the inner wall is unbroken, allowing fluid to pass through with minimal obstruction.

The sensor and chamber have fluid entry ports 540A and 540B, though the direction of fluid flow shown from left to right is an arbitrary choice. As mentioned above, in some marine sensors the fluid is pumped through the chamber; in others it may simply flow in and out without external influence, beyond the disturbance of the sensor itself being present. Possible fluid flow paths are indicated by interrupted dashed lines. In cases where a pump is used, inner circulation loops may occur as shown, with corresponding effects on the time the fluid may remain in contact with surface 530. In unpumped cases, efforts are made to minimize turbulent flow.

A free space UV beam is shown entering chamber 510 of sensor 500 from the left end of the figure. In the case shown, the shaded beam profile is a simplified representation of the beam as it first converges and then diverges as it travels axially. The objective, as discussed above with regard to FIG. 2, is to provide UV intensities close to the chamber's interior wall that can kill enough of the microorganisms that happen to be in the vicinity to either prevent biofilm formation altogether, or, more practically realizable, to keep their rate of growth low enough to significantly reduce the frequency at which sensor maintenance is required.

In the illustrated case, the minimum waist diameter of the UV beam after it enters the chamber is seen to be close to the chamber inlet port 540A. The axial position of the beam waist minimum diameter is determined by the design of the optical elements 404 and the positioning of BIS 400 relative to chamber 510, with the goal of delivering enough UV power to the chamber to provide sufficiently high beam intensities to the interior volume and surfaces of the chamber. In the figure, the surfaces of greatest interest—the top surfaces of the electrodes—are shown about halfway along the chamber axis, but such surfaces may be located elsewhere in a real sensor. The optical design of the elements within the BIS providing the beam may take axial shifts of the beam waist due to scattering in the fluid or at internal surfaces of the chamber. The UV beam produced by BIS 400 is subject to scattering losses in the fluid even before it enters the sensor chamber, as it passes through a significant volume of fluid between leaving housing 406 and reaching sensor 500.

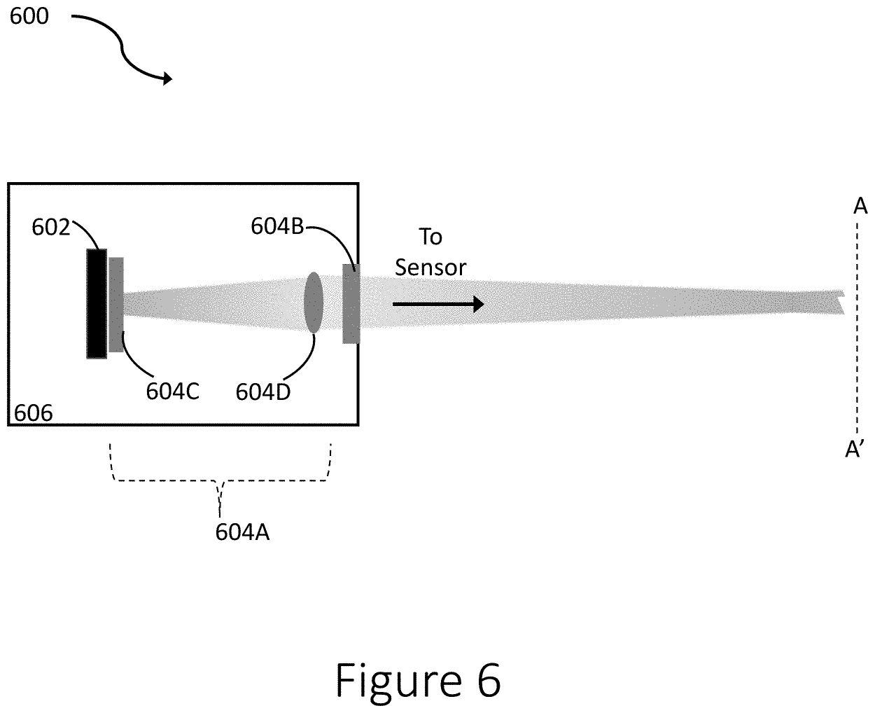

FIG. 6 illustrates an arrangement of the optical components of a BIS 600 that can provide a free space beam of the type shown in FIG. 5. As discussed above in discussing FIG. 2, the BIS to sensor separation must be at least equal to the diameter of the BIS housing, but as there is no flexible intervening optical fiber in the case of BIS 600, the housing window must be axially aligned with the entry port of the sensor. The beam shaping optics 604A have to be carefully selected and positioned to not only capture as much of the emission from UV-LED 602 as possible but also provide a beam exiting window 604B with an appropriate diameter and convergence characteristics to reach the sensor (not shown explicitly, but with the position of its nearest face indicated by dashed line AA′) with sufficient intensity to perform its intended function within the sensor chamber.

This objective may be achieved with the use of a pair of lenses, 604C and 604D, where 604C is a Fresnel lens and 604D is a conventional bulk lens. Some commercially available Fresnel lenses have been found to be extremely useful for this purpose, positioned very close to the UV-LED 602, and capturing a relatively large fraction of the UV light that may be emitted at extremely wide solid angles relative to the LED surface normal. Capture efficiency is close to 10×what is typically achieved using the conventional lens approach of fiber-guided embodiments. The combination of Fresnel lens 604C and conventional bulk lens 604D, can then focus much of the captured light into a beam that can exit the housing with the geometric “Gaussian” characteristics appropriate to reach the sensor and illuminate the sensor chamber as required.

A fused silica lens with a focal length between 20 mm and 100 mm is typically a good choice for lens 604D. In some embodiments in which a bare LED is available, rather the packaged LEDs as used in current experiments by the Applicants, a microlens (ball or half-ball) may be a good alternative to a Fresnel lens for lens 604C.

The two categories of BIS discussed above have different strengths and weaknesses, with the best choice for a particular application involving tradeoffs between factors such as spatial arrangement flexibility—how close the BIS must be or how far away the BIS may be positioned from the sensor, and whether it can be positioned off-axis—and UV capture and delivery efficiency The fiber-guided embodiments would often be favored by the first factor, while the free-space embodiments may have an edge for the second, but other factors like component cost, availability of special parts, ease of alignment, and durability under UV exposure will play a part too.

The two categories of BIS discussed above differ in the details of how UV radiation is processed and delivered to the sensor, but at a high level they operate to perform the same primary method steps—capturing UV light from a UV-LED source, optically processing the generated UV, and delivering the result to an entry port of the fluid-filled sensing chamber of a sensor such that the internal volume of that chamber is irradiated at sufficiently high UV intensity that biofilm formation and growth on one or more internal surfaces of interest within that sensor chamber are significantly inhibited. In each case, a watertight pressure resistance housing protects the majority or the entirety of the optical and electronic components of the BIS.

In both categories of BIS, the internal surfaces of the sensor chamber may be exposed to UV in bursts of very high intensity, or for longer periods, even continuously, at lower intensity, or in some combination of the two. The choice may depend on whether fluid is periodically pumped through the sensor chamber, or simply allowed to flow in and out naturally, as well as on local factors such as the concentration of micro-organisms expected to be present.

A different category of biofilm inhibition systems based on the same core idea of using UV to protect sensitive interior surfaces of marine sensors is made up of systems in which the fluid enters the sensor chamber after having been exposed to UV radiation from one or more UV-LEDs. In some of these embodiments, the exposure occurs in a separate chamber, specifically provided for that purpose, housing the one or more UV-LEDs, optional focusing optics, and any tubing or other mechanical components necessary to connect the chamber to the sensor so that the treated water can be pumped into the sensor chamber without contamination. In this way, the sensitive surfaces within the sensor will have minimal exposure to microorganisms present in the external fluid environment. It may be helpful to line the walls of the treatment chamber with a material that scatters UV for a more even illumination of fluid throughout the chamber volume. Plastic materials may be particularly useful in this regard in comparison to metals, which are much more susceptible to degradation when exposed to UV.

In some other embodiments of this third category of BIS, the exposure occurs laterally while the fluid is pumped through a relatively long tube to the sensor, using, for example, a side-emitting optical fiber carrying light from a UV source housed nearby. In yet other embodiments, exposure to the emission from multiple LEDs occurs in a chamber whose housing is attached directly to the sensor housing, the chamber's internal construction including features that maximize exposure efficiency by creating complex fluid flow paths and/or by providing highly reflective surfaces that mix the emissions of the point sources to distribute the light more uniformly through the moving fluid. Details of many of the embodiments in this third category are discussed and shown in the U.S. Provisional Patent Application referenced at the beginning of this disclosure.

It should be noted that in any of the BIS categories discussed above, more than one UV-LED may be required in particular applications, with corresponding additional optical components in the optical subsystem. In the first two categories discussed above, this may be done to provide sufficient UV illumination, or illumination distribution within, the sensor chamber to achieve the desired biofilm inhibition there. It may, for example, be helpful to deliver illumination into both inlet ports of a sensor chamber, and this may be relatively simple with a fiber-guided delivery approach. Multiple UV-LEDs may also be present for reliability/redundancy.

It should also be noted that for the sensor protection applications to which the present invention is directed, it is not necessary to provide UV intensities high enough to sterilize the fluid.

The present invention overcomes deficiencies and drawbacks of the prior art by using UV light to protect volumes and surfaces within chambers of marine sensors from biofilm formation and growth by irradiating either the chambers themselves, using optical fiber-guided or free space UV beams to address the interiors, or the fluid prior to entering the chambers. No toxic or otherwise hazardous materials are introduced into the marine environment. Power consumption is low, and the cost of the components is of the order of several hundreds of dollars, which should be easily offset by the anticipated saving in the frequency of maintenance trips to service the sensors. The potential cost savings in halving the number of visits to these sensors, which may be termed the “workhorses” of oceanographic research applications in remote and inhospitable locations, could be of the order of $1M per buoy array per year, each buoy having at least one sensor, and often more located at a range of depths along its mooring line.

While the discussion above has focused on marine sensors, it should be understood that marine applications, where the fluid of concern is seawater, are not the only applications for which the present invention may be useful. Such sensors are also used in freshwater environments where biofilms may be problematic. Other applications may also be envisaged, such as monitoring freshwater sources, monitoring aquaculture, sensors for defense applications, etc.

The present invention may be of interest in applications other than sensors containing interior chambers exposed to fluids, because with the appropriate choice of lenses in the systems discussed above, BIS embodiments discussed above may be adapted to inhibit biofouling on the windows of these or other types of sensors. Examples include cameras Standard or high definition), fluorometers (for biological measurements), spectral irradiance meter (for physical measurements), spectrophotometer (for biological measurements), and photosynthetically active radiation meters.

The embodiments described in this disclosure should be considered as illustrative examples of the present invention, rather than as limiting the scope of the invention. Various modifications of these embodiments will become apparent to those skilled in the art from the foregoing description and accompanying drawings.

1. A biofilm inhibition system (BIS) for a sensor comprising a sensor chamber with an interior volume and an interior surface exposed to direct contact with a fluid during sensor operation, the BIS comprising:
an ultraviolet light emitting diode (UV-LED); and
an optical subsystem coupled to the UV-LED to deliver a portion of the emitted UV light to illuminate a substantial fraction of the interior volume and the interior surface;
wherein a watertight housing encloses the UV-LED and at least a portion of the optical subsystem.

2. The BIS of claim 1, wherein the UV-LED is a UVC-LED, emitting light of wavelength between 200 nm and 300 nm.

3. The BIS of claim 1,
wherein the interior surface comprises an electrode surface;
wherein the optical subsystem is configured to deliver the portion of the emitted UV light through an inlet port of the sensor chamber; and
wherein the watertight housing is capable of withstanding external pressure up to a predetermined threshold determined by an anticipated maximum operating depth for the sensor.

4. The BIS of claim 3, wherein the anticipated maximum operating depth is 200 m.

5. The BIS of claim 3,
wherein the sensor is a marine conductivity sensor; and
wherein the fluid is seawater.

6. The BIS of claim 1,
wherein the optical subsystem comprises:
a lens, capturing emission from the UV-LED; and
a UV-transparent optical fiber, having an input end positioned to receive the captured emission from the UV-LED within, or at a boundary surface of, the watertight housing, and an output end, outside the waterproof housing, positioned to deliver a portion of the emitted UV light through an inlet port of the sensor chamber to illuminate the interior volume and the interior surface.

7. The BIS of claim 1,
wherein no part of the optical subsystem is present outside the waterproof housing; and
wherein the optical subsystem comprises a Fresnel lens and a conventional lens, positioned such that the Fresnel lens captures light emitted by the UV-LED, and the conventional lens captures the output from the Fresnel lens to form a free space beam, the beam emerging from a transparent window in the waterproof housing and configured to reach an inlet port of the sensor chamber to illuminate the interior volume and the interior surface.

8. The BIS of claim 7, wherein the free space beam formed by the BIS is characterized by a beam waist that reaches a minimum value before entering the inlet port of the sensor chamber.

9. The BIS of claim 1,
wherein no part of the optical subsystem is present outside the waterproof housing; and
wherein the optical subsystem comprises a ball or half-ball microlens and a conventional lens, positioned such that the microlens captures light emitted by the UV-LED, and the conventional lens captures the output from the microlens to form a free space beam, the beam emerging from a transparent window in the waterproof housing and configured to reach an inlet port of the sensor chamber to illuminate the interior volume and the interior surface.

10. A biofilm inhibition system (BIS) for a sensor comprising a sensor chamber with an interior surface exposed to direct contact with a fluid during sensor operation, the BIS comprising:
a fluid treatment chamber, having an input port for the entry of untreated fluid and an output port from which treated fluid may be pumped from the treatment chamber to enter the sensor chamber; and
an ultraviolet light emitting diode (UV-LED) configured to illuminate the fluid within the fluid treatment chamber;
wherein a watertight housing encloses the UV-LED.

11. The BIS of claim 10, additionally comprising an optical subsystem coupled to the UV-LED to enhance UV illumination of the fluid.

12. The BIS of claim 10,
wherein the watertight housing is capable of withstanding external pressure up to a predetermined threshold determined by an anticipated maximum operating depth for the sensor;
wherein the sensor is a marine conductivity sensor;
wherein the interior surface comprises an electrode surface; and
wherein the fluid is seawater.

13. A method of inhibiting development of a biofilm in a sensor comprising a sensor chamber with an interior volume and an interior surface exposed to direct contact with a fluid during sensor operation; the method comprising;
capturing emission from an ultraviolet light emitting diode (UV-LED); and
using an optical subsystem coupled to the UV-LED to deliver a portion of the emitted UV light to illuminate a substantial fraction of the interior volume and the interior surface;
wherein a watertight housing encloses the UV-LED and at least a portion of the optical subsystem.

14. The method of claim 13, wherein the UV-LED is a UVC-LED, emitting light of wavelength between 200 nm and 300 nm.

15. The method of claim 13,
wherein the delivery of the portion of the emitted UV light occurs through an inlet port of the chamber; and
wherein the watertight housing is capable of withstanding external pressure up to a predetermined threshold determined by an anticipated maximum operating depth for the sensor.

16. The method of claim 15,
wherein the sensor is a marine conductivity sensor;
wherein the interior surface comprises an electrode surface; and
wherein the fluid is seawater.

17. The method of claim 13,
wherein the optical subsystem of the BIS comprises:
a lens, capturing emission from the UV-LED; and
a UV-transparent optical fiber, having an input end positioned to receive the captured emission from the UV-LED within, or at a boundary surface of, the watertight housing, and an output end, outside the waterproof housing, positioned to deliver a portion of the emitted UV light through an inlet port of the chamber to illuminate the interior volume and the interior surface of the chamber.

18. The method of claim 17,
wherein no part of the optical subsystem is present outside the waterproof housing; and
wherein the optical subsystem comprises a Fresnel lens and a conventional lens, positioned such that the Fresnel lens captures light emitted by the UV-LED, and the conventional lens captures the output from the Fresnel lens to form a free space beam, the beam emerging from a transparent window in the waterproof housing and configured to reach an inlet port of the chamber to illuminate the interior volume and the interior surface of the chamber.

19. The method of claim 18, wherein the free space beam formed by the BIS is characterized by a beam waist that reaches a minimum value before entering the inlet port of the sensor chamber.

19. The method of claim 17,
wherein no part of the optical subsystem is present outside the waterproof housing; and
wherein the optical subsystem comprises a ball or half-ball microlens and a conventional lens, positioned such that the microlens captures light emitted by the UV-LED, and the conventional lens captures the output from the microlens to form a free space beam, the beam emerging from a transparent window in the waterproof housing and configured to reach an inlet port of the sensor chamber to illuminate the interior volume and the interior surface.
